(19) United States
(12) Patent Application Publication
(10) Pub. No.: US 2023/0323460 A1
(43) Pub. Date: Oct. 12, 2023

(54) DETECTION AND TREATMENT OF AUTISM SPECTRUM DISORDERS

(71) Applicant: Seawire Consultoria e Comercio LDA, Macao (CN)

(72) Inventor: Paul Yao, Wantagh, NY (US)

(21) Appl. No.: 18/176,360

(22) Filed: Feb. 28, 2023

Related U.S. Application Data
(63) Continuation of application No. 16/848,494, filed on Apr. 14, 2020, now abandoned.

(30) Foreign Application Priority Data
Dec. 13, 2019 (WO) PCT/CN2019/125335

(51) Int. Cl. C12Q 1/6883

(52) U.S. Cl. CPC C12Q 1/6883; A61K 45/06; G01N 2333/726; G01N 2800/50

(57) ABSTRACT

The present disclosure relates generally to compositions and methods for diagnosing or predicting autism spectrum disorder (ASD). The methods are based on the discovery that certain genes, such as SOD2, OXT, GPER, and ERRα, have different expression levels in CD34+ cells or MNCs in ASD patients as compared to individuals not having ASD. Once the ASD patient is identified or predicted, compositions and methods are also provided for preventing or treating the ASD.

### CROSS-REFERENCE TO RELATED APPLICATIONS

This application is a continuation of U.S. patent application Ser. No. 16/848,494, filed Apr. 14, 2020, which is a continuation application under 35 U.S.C. § 111(a) of the International Application No. PCT/CN2019/125335, filed Dec. 13, 2019, the contents of which are incorporated herein by reference in their entireties.

### REFERENCE TO AN ELECTRONIC SEQUENCE LISTING

The contents of the electronic sequence listing (69AA-305409-US2.xml; Size: 33,632 bytes; and Date of Creation: Feb. 28, 2023) is herein incorporated by reference in its entirety.

### BACKGROUND

Autism spectrum disorder (ASD) is a group of developmental disabilities that include full syndrome autism, Asperger's syndrome, and other pervasive developmental disorders. ASD is now diagnosed in more than 1 out of 59 children, and ASD prevalence has been increasing continuously. ASD is inexplicably biased towards males by a ratio of at least 4:1.

ASD can be detected as early as 18 months or even younger in some cases. A reliable diagnosis can usually be made by the age of two years. The diverse expressions of ASD symptoms pose diagnostic challenges to clinicians. Individuals with an ASD may present at various times of development (e.g., toddler, child, or adolescent), and symptom expression may vary over the course of development. Furthermore, clinicians must differentiate among pervasive developmental disorders, and may also consider similar conditions, including intellectual disability not associated with a pervasive developmental disorder, specific language disorders, ADHD, anxiety, and psychotic disorders.

Considering the unique challenges in diagnosing ASD, specific practice parameters for its assessment have been published by the American Academy of Neurology, the American Academy of Child and Adolescent Psychiatry, and a consensus panel with representation from various professional societies. The practice parameters outlined by these societies include an initial screening of children by general practitioners and for children who fail the initial screening, a comprehensive diagnostic assessment by experienced clinicians. Furthermore, it has been suggested that assessments of children with suspected ASD be evaluated within a developmental framework, include multiple informants (e.g., parents and teachers) from diverse contexts (e.g., home and school), and employ a multidisciplinary team of professionals (e.g., clinical psychologists, neuropsychologists, and psychiatrists).

Many possible explanations and potential causative factors have been reported, such as genetic, epigenetic and environmental factors, while the detailed mechanisms of ASD remain unclear. Risk factors include having an older parent, a family history of autism, and certain genetic conditions. Diagnosis is based on symptoms. The DSM-5 redefined the autism spectrum disorders to encompass the previous diagnoses of autism, Asperger syndrome, pervasive developmental disorder not otherwise specified (PDD-NOS), and childhood disintegrative disorder.

Treatment efforts are generally individualized, and can include behavioral therapy, and the teaching of coping skills. Medications may be used to try to help improve symptoms. Evidence to support the use of medications, however, is not very strong.

### SUMMARY

The instant invention made the discovery that certain genes are consistently up- or down-regulated in CD34+ cells or Mononuclear cells (MNCs) isolated from the peripheral blood or umbilical cord blood of individuals having ASD as compared to those not having ASD. These genes include SOD2 (superoxide dismutase 2), OXT (oxytocin/neurophysin I prepropeptide), GPER (G protein-coupled estrogen receptor 1), and ERRα (estrogen related receptor α), which under-expressed in ASD samples.

Accordingly, the present disclosure provides methods and compositions for predicting or diagnosing ASD based on expression measurement of one or more such genes in a patient. Further, it is contemplated that restoring the normal expression of some of these genes can be helpful in treating ASD or ameliorating some of the ASD symptoms, especially when the restoration is made in affected neural cells.

### BRIEF DESCRIPTION OF THE DRAWINGS

FIG. 1. The expression of ERβ and ERβ-related genes were decreased in ASD patients. 3 ml of peripheral blood was withdrew from either control (CTL) or ASD children (2-6 years old), and the mononuclear cells (MNCs) were isolated and the mRNA was purified, and mRNA expression was evaluated by real time PCR. n=61 (for CTL) and 64 (for ASD). *, P<0.05, vs CTL group; ¶, P<0.01, vs CTL group.

FIG. 2. The ROC curve based on the expression of SOD2 from both CTL and ASD patients. The SOD2 mRNA expression levels from both CTL (n=61) and ASD (n=64) patients were used to draw the ROC (receiver operating characteristic) curve. (a) Case processing summary, positive means CTL cases, and negative means ASD cases. (b) ROC curve. (c) Area under the curve.

FIG. 3. Establishment of Cut/Off Value for the diagnosis of ASD patients based on SOD2 mRNA expression. (a) The coordinates of the curve, part a. (b) The coordinates of the curve, part b. (c) The coordinates of the curve, part c.

### DETAILED DESCRIPTION

### Definitions

The following description sets forth exemplary embodiments of the present technology. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

As used herein, certain terms may have the following defined meanings. As used in the specification and claims, the singular form “a,” “an” and “the” include singular and plural references unless the context clearly dictates otherwise. For example, the term “a cell” includes a single cell as well as a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

The disclosure further provides diagnostic, prognostic and therapeutic methods, which are based, at least in part, on determination of the expression level of a gene of interest identified herein.

For example, information obtained using the diagnostic assays described herein is useful for determining if a subject is likely suffering from a disease (e.g., ASD) or likely to develop the disease, or is suitable for a treatment. Based on the diagnostics/prognostic information, a doctor can recommend a therapeutic protocol.

It is to be understood that information obtained using the diagnostic assays described herein may be used alone or in combination with other information, such as, but not limited to, behavior assessment, genotypes or expression levels of other genes, clinical chemical parameters, histopathological parameters, or age, gender and weight of the subject.

### Diagnostic Prognosis Methods, and Computer Program Products

The diagnosis of autism spectrum disorder (ASD) is challenging, primarily relying on behavior assessment, which is disruptive and inaccurate. Meanwhile, because the molecular mechanism is unclear, there is no reliable diagnosis on the molecular or clinical chemistry level. The instant inventor made the surprising discovery that certain genes are consistently down-regulated in the CD34+ or MNCs cells isolated from the blood of ASD patients as compared to healthy control individuals. Accordingly, the expression levels of these genes can be used to diagnose or predict ASD. It is also contemplated that the restoration of the expression of one or more of these genes can be helpful in treating ASD or one or more of its symptoms.

In accordance with one embodiment of the present disclosure, provided is a method for identifying gene expression information useful for diagnosing or predicting an autism spectrum disorder (ASD). In some embodiments, the method entails measuring the expression level of one or more genes selected from the group consisting of SOD2 (superoxide dismutase 2), OXT (oxytocin/neurophysin I prepropeptide), GPER (G protein-coupled estrogen receptor 1), and/or ERRα (estrogen related receptor alpha). Preferably, the expression level is detected in MNC cells, or CD34+ cells.

### Table A. Listing of Genes Tested in ASD Patients

TABLE A
Listing of Genes Tested in ASD Patients

| Gene | Full name | Accession # |
|---|---|---|
| SOD2 | superoxide dismutase 2 | NM_000636 |
| ERβ | estrogen receptor 2 | NM_001040275 |
| OXT | oxytocin/neurophysin I prepropeptide | NM_000915 |
| GPER | G protein-coupled estrogen receptor 1 | NM_001505 |
| ERα | estrogen receptor 1 | NM_000125 |
| ERRα | estrogen related receptor alpha | NM_004451 |
| AR | androgen receptor | NM_000044 |
| VDR | vitamin D receptor | NM_000376 |
| PGR | progesterone receptor | NM_001202474 |
| RORA | retinoic acid-related orphan receptor α | NM_134261 |
| OXTR | oxytocin receptor | NM_000916 |

In some embodiments, the measurement is done for one or more genes selected from the group consisting of SOD2, OXT, GPER, and ERRα. In some embodiments, the measurement is done for one or more genes selected from the group consisting of SOD2, OXT, and GPER. In some embodiments, the measurement is done for one or more genes selected from the group consisting of SOD2, OXT, and ERRα. In some embodiments, the measurement is done for one or more genes selected from the group consisting of SOD2, GPER, and ERRα. In some embodiments, the measurement is done for one or more genes selected from the group consisting of SOD2, and OXT. In some embodiments, the measurement is done for one or more genes selected from the group consisting of SOD2, and GPER. In some embodiments, the measurement is done for one or more genes selected from the group consisting of SOD2, and ERRα. In some embodiments, the measurement is done for at least SOD2.

Once the expression level of one or more of these genes is detected, it can be compared to suitable reference levels to determine whether it is increased or decreased. It would be readily appreciated by the skilled artisan that the increase or decrease of an expression level or a ratio are relative terms but can be readily ascertained.

In one aspect, an “internal control” can be used to normalize the measurement to correct sample collection variations. One such internal control is a “housekeeping” gene that refers to any constitutively or globally expressed gene. Examples of such genes include, but are not limited to, β-actin, the transferring receptor gene, GAPDH gene or equivalents thereof. In one aspect of the disclosure, the internal control gene is β-actin. In one of aspect of the disclosure, the internal control gene is GAPDH.

Normalized expression levels or ratios can then be compared to a suitable control sample. In one aspect, the control sample is a sample collected from a non-diseased subject or a non-diseased sample from the same subject. In some aspects, a suitable control sample is not necessarily from a particular individual, but can be a “virtual control” sample which has the average value of a group of control samples. In some embodiments, the control is selected based on certain characteristics of the test subject. For instance, a suitable control sample would be from an individual having the same gender, similar age, or body weight, without limitation.

In some embodiments, the term “overexpression” or “underexpression” refers to increased or decreased expression, or alternatively a differential expression, of a gene in a test sample as compared to the expression level of that gene in the control sample. In one aspect, the differential expression is at least about 5%, or alternatively at least about 10%, 15%, 20%, 25%, 30%, 35%, 40%, 45%, 50%, 55%, 60%, 65%, 70%, 75%, 80%, 85%, 90%, 95%, or at least about 2 times, 3 times, or 5 times higher or lower than the expression level detected in the control sample. Alternatively, the gene is referred to as “over expressed” or “under expressed”. Alternatively, the gene may also be referred to as “up regulated” or “down regulated”.

In an alternative scenario, the increase or decrease can be compared to a “predetermined value” that separates two different states. A “predetermined value” for a gene as used herein, is so chosen that a patient with an expression level of that gene higher than (or lower than, as the case may be) the predetermined value is likely to suffer (or likely to develop) ASD. One of skill in the art can determine a predetermined value for a gene by comparing expression levels of a gene in ASD patients to those without ASD. In one aspect, a predetermined value is a gene expression value that best separates patients into a group with ASD and a group without ASD. Such a gene expression value can be mathematically or statistically determined with methods known in the art.

In some embodiments, a decreased expression level of SOD2, OXT, GPER, or ERRα identifies the human patient as likely suffering from ASD or likely to develop ASD. In some embodiments, a decreased expression level of SOD2 identifies the human patient as likely suffering from ASD or likely to develop ASD. In some embodiments, a decreased expression level of OXT identifies the human patient as likely suffering from ASD or likely to develop ASD. In some embodiments, a decreased expression level of GPER identifies the human patient as likely suffering from ASD or likely to develop ASD. In some embodiments, a decreased expression level of ERRα identifies the human patient as likely suffering from ASD or likely to develop ASD.

In some embodiments, a threshold is established for evaluating the differential expression levels. For instance, in some embodiments, a decreased expression level of SOD2 of more than 1, 5, 2, 2.5, 3, 3.5, 4, 4.5, 5, 6, 7, 8, 9, or 10 fold identifies the human patient as likely suffering from ASD or likely to develop ASD. In some embodiments, a decreased expression level of SOD2 of more than 95%, 90%, 85%, 80%, 75%, 70%, 65%, 60%, 55% or 50% identifies the human patient as likely suffering from ASD or likely to develop ASD.

In some embodiments, a decreased expression level of GPER of more than 1, 5, 2, 2.5, 3, 3.5, 4, 4.5, 5, 6, 7, 8, 9, or 10 fold identifies the human patient as likely suffering from ASD or likely to develop ASD. In some embodiments, a decreased expression level of GPER of more than 95%, 90%, 85%, 80%, 75%, 70%, 65%, 60%, 55% or 50% identifies the human patient as likely suffering from ASD or likely to develop ASD.

In some embodiments, a decreased expression level of OXT of more than 1, 5, 2, 2.5, 3, 3.5, 4, 4.5, 5, 6, 7, 8, 9, or 10 fold identifies the human patient as likely suffering from ASD or likely to develop ASD. In some embodiments, a decreased expression level of OXT of more than 95%, 90%, 85%, 80%, 75%, 70%, 65%, 60%, 55% or 50% identifies the human patient as likely suffering from ASD or likely to develop ASD.

In some embodiments, a decreased expression level of ERRα of more than 1, 5, 2, 2.5, 3, 3.5, 4, 4.5, 5, 6, 7, 8, 9, or 10 fold identifies the human patient as likely suffering from ASD or likely to develop ASD. In some embodiments, a decreased expression level of ERRα of more than 95%, 90%, 85%, 80%, 75%, 70%, 65%, 60%, 55% or 50% identifies the human patient as likely suffering from ASD or likely to develop ASD.

In some embodiments, a combination of changes of expression of levels of two or more of the genes is used. In some embodiments, a combination of changes of expression of levels of three or more of the genes is used. In some embodiments, a combination of changes of expression of levels of four or more of the genes is used.

In some embodiments, the decrease of expression levels of both SOD2 and ERRα identifies the human patient as likely suffering from ASD or likely to develop ASD. In some embodiments, the combination is the decrease of expression of SOD2 and OXT, or SOD2 and GPER, or ERRα and OXT, ERRα and GPER, or OXT and GPER.

In some embodiments, the combination is the decrease of SOD2 and ERRα or OXT, SOD2 and ERRα or GPER, SOD2 and OXT or GPER, SOD2 and one or two of ERRα, OXT or GPER.

The gene expression, as referred herein, may be measured at protein level or mRNA level, without limitation. Methods for measuring protein and mRNA levels of a gene in a cell are well known in the art. For the purpose of illustration only, such methods can include determining the amount of a mRNA transcribed from the gene using, for example, in situ hybridization, PCR, real-time PCR, or microarray. Methods of determining protein expression levels are also known in the art, such as, without limitation, western blotting, immunohistochemistry, ELISA or protein microarrays.

The gene expression, in some embodiments, is measured in a CD34+ cell isolated from a blood sample, or a sample enriched with CD34+ cells. The method, therefore, further entails isolating MNC or CD34+ cells from the patient prior to gene expression measurement. The MNC or CD34+ cell may be isolated from peripheral or umbilical cord blood.

Methods of isolating MNC and CD34+ cells are exemplified in the attached experimental examples. In some embodiments, the samples are freshly prepared. For instance, the measurement is started within 8, 7, 6, 5, 4, 3, or 2 hours following isolation of the cell from the human patient.

It is preferable that the sample used for gene expression measurement is enriched in MNC cells. In some embodiments, the sample includes a cell population that includes at least 50%, or preferably at last 75%, 85%, 90% or 95% CD34+ cells. In some embodiments, the sample includes a cell population that includes at least 50%, or preferably at least 75%, 85%, 90% or 95% MNC cells.

In another embodiment, the present invention is a computer program product for use in conjunction with a computer system, the computer program product comprising a computer readable storage medium and a computer program mechanism embedded therein, the computer mechanism comprising executable instructions for performing a method for identifying gene expression information useful for diagnosing or predicting an autism spectrum disorder (ASD), wherein the instructions comprising: (i) obtaining measuring the measurement of the expression level of one or more genes selected from the group consisting of SOD2 (superoxide dismutase 2), OXT (oxytocin/neurophysin I prepropeptide), GPER (G protein-coupled estrogen receptor 1), and ERRα (estrogen related receptor alpha) in a mononuclear cell (MNC) or a CD34+ cell isolated from a human patient; and (ii) comparing the measurement of the expression level to a reference expression level from a corresponding cell from a reference human subject not suffering from an ASD, wherein a decreased expression level of SOD2, OXT, GPER, or ERRα identifies the human patient as likely suffering from ASD or likely to develop ASD.

In some embodiments, wherein a decreased expression of more than 50% identifies the human patient as suffering from ASD or likely to develop ASD.

In some embodiments, wherein the reference human subject is an actual reference human subject, or a virtual reference human subject created with pool data from one or more human subjects not suffering from an ASD.

### Treatment Methods and Compositions

Compositions and methods of preventing and treating ASD are also provided, which can be employed once a patient is identified as likely to have ASD or to develop ASD.

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms.

“Prevention” or “preventing” means any treatment of a disease or condition that causes the clinical symptoms of the disease or condition not to develop. Compositions may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of ASD. The therapeutically effective amount may vary depending on the subject, and disease or condition being treated, the weight and age of the subject, the severity of the disease or condition, and the manner of administering, which can readily be determined by one or ordinary skill in the art.

There are certain known therapies for ASD, including behavior treatments, medication and nutrition. For instance, Applied Behavior Analysis (ABA) is often used in schools and clinics to help your child learn positive behaviors and reduce negative ones. This approach can be used to improve a wide range of skills, and there are different types for different situations, including discrete trial training (DTT) which uses simple lessons and positive reinforcement. Pivotal response training (PRT) helps develop motivation to learn and communicate. Early intensive behavioral intervention (EIBI) is best for children under age 5. Verbal behavior intervention (VBI) focuses on language skills.

Developmental, Individual Differences, Relationship-Based Approach (DIR) is a treatment that is better known as Floortime. DIR supports emotional and intellectual growth by helping him learn skills around communication and emotions.

Treatment and Education of Autistic and Related Communication-handicapped Children (TEACCH) uses visual cues such as picture cards to help your child learn everyday skills like getting dressed. Information is broken down into small steps so he can learn it more easily.

The Picture Exchange Communication System (PECS) is a visual-based treatment, but it uses symbols instead of picture cards. The child learns to ask questions and communicate through special symbols.

Occupational Therapy is also available which helps a child learn life skills like feeding and dressing himself, bathing, and understanding how to relate to other people. The skills he learns are meant to help him live as independently as he can. Sensory Integration Therapy can help a child learn to deal with that kind of sensory information.

There is no cure for autism spectrum disorder, and there's currently no medication to treat it. But some medicines can help with related symptoms like depression, seizures, insomnia, and trouble focusing. Risperidone (Risperdal) is the only drug approved by the FDA for children with autism spectrum disorder. It can be prescribed for children between 5 and 16 years old to help with irritability. Some doctors will prescribe other drugs in certain cases, including selective serotonin reuptake inhibitors (SSRIs), anti-anxiety medications, or stimulants, but they're not FDA-approved for autism spectrum disorder.

New treatments are contemplated in the present disclosure. In some embodiments, the treatment entails administering to a patient an agent that increases the biological activity of SOD2, OXT, GPER, or ERRα in the patient. The increase may occur in any cell of the patient, such as blood cells, but preferably the increase or decrease occurs in a neuron.

Methods are known to prepare agents that can increase or decrease the biological activity of a gene. An agent that decreases the biological activity of a gene can be an antagomir, an antibody, a siRNA, a ribozyme, without limitation. Antagomirs are a class of chemically engineered oligonucleotides and can be used to silence endogenous microRNA.

Methods for increasing the level of a protein, or polypeptide or peptide, such as SOD2, OXT, GPER, or ERRα, in a cell are known in the art. In one aspect, the gene level is increased by increasing the amount of a polynucleotide encoding gene, as provided above, wherein that polynucleotide is expressed such that new gene is produced. In another aspect, increasing the gene level is increased by increasing the transcription of a polynucleotide encoding gene, or alternatively translation of gene, or alternatively post-translational modification, activation or appropriate folding of gene. In yet another aspect, increasing gene level is increased by increasing the binding of the protein to appropriate cofactor, receptor, activator, ligand, or any molecule that is involved in the protein's biological functioning. In some embodiments, increasing the binding of gene to the appropriate molecule is increasing the amount of the molecule. In one aspect of the embodiments, the molecule is the gene protein. In another aspect of the embodiments, the molecule is a small molecule. In a further aspect of the embodiments, the molecule is a polynucleotide.

Methods of increasing the amount of polynucleotide in a cell are known in the art and can be modified for increasing the amount of a polynucleotide encoding the gene. In one aspect, the polynucleotide can be introduced to the cell and expressed by a gene delivery vehicle that can include a suitable expression vector. Change of DNA methylation or acetylation can also be used to regulate the expression of a target gene. In some embodiments, the expression of a gene of the present disclosure can be increased by inhibiting methylation of its promoter or regulatory regions.

Suitable expression vectors are well-known in the art, and include vectors capable of expressing a polynucleotide operatively linked to a regulatory element, such as a promoter region and/or an enhancer that is capable of regulating expression of such DNA. Thus, an expression vector refers to a recombinant DNA or RNA construct, such as a plasmid, a phage, recombinant virus or other vector that, upon introduction into an appropriate host cell, results in expression of the inserted DNA. Appropriate expression vectors include those that are replicable in eukaryotic cells and/or prokaryotic cells and those that remain episomal or those which integrate into the host cell genome.

As used herein, the term “vector” refers to a non-chromosomal nucleic acid comprising an intact replicon such that the vector may be replicated when placed within a cell, for example by a process of transformation. Vectors may be viral or non-viral. Viral vectors include retroviruses, adenoviruses, herpesvirus, papovirus, or otherwise modified naturally occurring viruses. Exemplary non-viral vectors for delivering nucleic acid include naked DNA; DNA complexed with cationic lipids, alone or in combination with cationic polymers; anionic and cationic liposomes; DNA-protein complexes and particles comprising DNA condensed with cationic polymers such as heterogeneous polylysine, defined-length oligopeptides, and polyethylene imine, in some cases contained in liposomes; and the use of ternary complexes comprising a virus and polylysine-DNA.

Non-viral vector may include plasmid that comprises a heterologous polynucleotide capable of being delivered to a target cell, either in vitro, in vivo or ex-vivo. The heterologous polynucleotide can comprise a sequence of interest and can be operably linked to one or more regulatory elements and may control the transcription of the nucleic acid sequence of interest. As used herein, a vector need not be capable of replication in the ultimate target cell or subject. The term vector may include expression vector and cloning vector.

Gene delivery vehicles also include DNA/liposome complexes, micelles and targeted viral protein-DNA complexes. Liposomes that also comprise a targeting antibody or fragment thereof can be used in the methods of this invention. To enhance delivery to a cell, the nucleic acid or proteins of this invention can be conjugated to antibodies or binding fragments thereof which bind cell surface antigens, e.g., a cell surface marker found on stem cells or cardiomyocytes. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this invention are other non-limiting techniques.

The agents can be administered by any suitable formulation. Accordingly, a formulation comprising the necessary therapy is further provided herein. The formulation can further comprise one or more preservatives or stabilizers. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, and 1.0%).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional proviso that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

Various delivery systems are known and can be used to administer a chemotherapeutic agent of the disclosure, e.g., encapsulation in liposomes, microparticles, microcapsules, expression by recombinant cells, receptor-mediated endocytosis. See e.g., Wu and Wu (1987) J. Biol. Chem. 262:4429-4432 for construction of a therapeutic nucleic acid as part of a retroviral or other vector, etc. Methods of delivery include but are not limited to intra-arterial, intra-muscular, intravenous, intranasal and oral routes. In a specific embodiment, it may be desirable to administer the pharmaceutical compositions of the disclosure locally to the area in need of treatment; this may be achieved by, for example, and not by way of limitation, local infusion during surgery, by injection or by means of a catheter.

The agents identified herein as effective for their intended purpose can be administered to subjects or individuals identified by the methods herein as suitable for the therapy. Therapeutic amounts can be empirically determined and will vary with the pathology being treated, the subject being treated and the efficacy and toxicity of the agent.

Methods of administering pharmaceutical compositions are well known to those of ordinary skill in the art and include, but are not limited to, oral, microinjection, intravenous or parenteral administration. The compositions are intended for topical, oral, or local administration as well as intravenously, subcutaneously, or intramuscularly. Administration can be effected continuously or intermittently throughout the course of the treatment. Methods of determining the most effective means and dosage of administration are well known to those of skill in the art and will vary with the ASD being treated and the patient and the subject being treated. Single or multiple administrations can be carried out with the dose level and pattern being selected by the treating physician.

### Kits and Packages

The methods described herein may be performed, for example, by utilizing pre-packaged diagnostic kits, such as those described below, comprising at least one probe or primer nucleic acid described herein, which may be conveniently used, e.g., to determine whether a subject has or is at risk of developing a disease such as ASD.

Diagnostic procedures can be performed with mRNA isolated from cells or in situ directly upon tissue sections (fixed and/or frozen) of primary tissue such as biopsies obtained from biopsies or resections, such that no nucleic acid purification is necessary. Nucleic acid reagents can be used as probes and/or primers for such in situ procedures (see, for example, Nuovo, G. J. (1992) PCR IN SITU HYBRIDIZATION: PROTOCOLS AND APPLICATIONS, RAVEN PRESS, NY).

In addition to methods which focus primarily on the detection of one nucleic acid sequence, profiles can also be assessed in such detection schemes. Fingerprint profiles can be generated, for example, by utilizing a differential display procedure, Northern analysis and/or RT-PCR.

In one embodiment, provided is a kit or package useful for diagnosing ASD, comprising nucleotide primers or probes for measuring the mRNA expression level of one or more genes selected from the group consisting of SOD2, OXT, GPER, and ERRα. In some embodiments, the primers or probes are useful for measuring at least two of the genes, such as SOD2, OXT, GPER, and ERRα, SOD2, OXT, and GPER, SOD2, OXT, and ERRα, SOD2, GPER, and ERRα, SOD2, and OXT, SOD2, and GPER, or SOD2, and ERRα.

In some embodiments, the primers or probes are useful for measuring at least one, two or three genes of SOD2, OXT, GPER, and ERRα. In some embodiments, the primers or probes are useful for measuring at least one, two or three genes of SOD2, OXT, GPER, and ERRα.

The kit or package may also be for measuring the protein expression. For instance, one embodiment provides a kit or package useful for diagnosing ASD, comprising antibodies for measuring the protein expression level of one or more genes selected from the group consisting of SOD2, OXT, GPER, and ERRα. In some embodiments, the antibodies are useful for measuring at least two of the genes, such as SOD2, OXT, GPER, and ERRα, SOD2, OXT, and GPER, SOD2, OXT, and ERRα, SOD2, GPER, and ERRα, SOD2, and OXT, SOD2, and GPER, or SOD2, and ERRα.

The kit or package, in some embodiments, can also include a marker for identifying MNC or CD34+ cells. An example marker is an anti-CD34 antibody. Also included, in some embodiments, is a container containing an anticoagulant for collecting a blood sample. Also included, in some embodiments, is a stem cell culture medium for culturing a CD34+ or MNC cell. Also provided, in the kit or package, are reagents for purifying mRNA, protein, cells, without limitation.

In one embodiment, a kit further includes instructions for use. In one aspect, a kit includes a manual comprising reference gene expression levels.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Identification of Genes Down- or Up-Regulated in CD34+ Cells

Preliminary large-scale screening experiments were conducted to show that certain genes were considerably over- or under-expressed in CD34+ cells isolated from peripheral blood or umbilical cord blood of patients with autism spectrum disorder (ASD), as compared to individuals not having ASD. This example confirms the findings with RT-PCR. The genes of concern included SOD2 (superoxide dismutase 2), ERβ (estrogen receptor (3), OXT (oxytocin/neurophysin I prepropeptide), GPER (G protein-coupled estrogen receptor 1), ERα (estrogen receptor α), ERRα (estrogen related receptor α), AR (androgen receptor), VDR (vitamin D receptor), PGR (progesterone receptor), RORA and OXTR (oxytocin receptor).

Collection of Peripheral or Umbilical Cord Blood. Peripheral blood samples (3-5 ml) from <4-year old children or umbilical cord blood (3-200 ml) from newborns were collected using EDTA (preferred), Heparin, citrate, acid citrate dextrose (ACD), or citrate phosphate dextrose (CPD formulation: Citric acid 3.27 g, Trisodium citrate 26.3 g, Sodium phosphate 2.51 g, Glucose 25.5 g, qs to 1000 mL) solution as anticoagulants. The related Institutional Review Board approved the protocol for Ethical Approval and Consent to participate, and informed consent were obtained from all donors and parents of newborns. The collected blood samples were refrigerated at 4° C. immediately after collection. They could also be shipped in refrigerated packaging at 4° C. The blood samples were used for either isolation of mononuclear cells or CD34 positive stem cells as soon as possible in maximum 6 hours.

Isolation of Mononuclear Cells (MNCs). All glassware that came in contact with the samples were siliconized before use. Glassware were immersed in a 1% silicone solution (Sigmacote, #SL2, from Sigma Chemicals) for 10 seconds, washed thoroughly with distilled water, and dried in an oven. Mononuclear cells (MNCs) were isolated from fresh blood using Lymphoprep™ reagents (#07861, from STEMCELL Technologies). Lymphoprep™ is the density gradient medium for the isolation of MNCs. The blood volume was between 3-15 ml. The blood samples were processed at temperatures of 15-25° C. The isolated MNCs were used for isolation of CD34 positive stem cells, or cryopreserved for future usage.

Isolation of CD34 Positive Stem Cells. CD34 positive stem cells were isolated from either freshly prepared or previously frozen MNCs using the EasySep™ Human CD34 Positive Selection Kit (#18056, from STEMCELL Technologies). When using previously frozen MNCs, the cells were incubated with DNase I Solution (#07900, from STEMCELL Technologies) at a concentration of 100 μg/mL at room temperature (15-25° C.) for at least 15 minutes prior to labeling and separation. Aggregated suspensions were filtered through a 40 μm Cell Strainer (#27305, from STEMCELL Technologies) for optimal results. When isolating CD34+ cells from fresh cord blood, the EasySep™ Human Cord Blood CD34 Selection Kit II (#17896, STEMCELL Technologies) was used. When isolating CD34+ cells from fresh whole blood, the EasySep™ Whole Blood CD34 Selection Kit (#18086, STEMCELL Technologies) were used. After preparation, the cells were resuspended in recommended medium for further expansion or directly use them for isolation of mRNA using the RNeasy Plus Mini Kit (Qiagen).

Assessment of the purity of CD34 positive cells. The isolated CD34 positive cells were used for assessment of purity by flow cytometry. The cells were labeled using anti-Human CD34 Antibody, Clone 581 (#60013, STEMCELL Technologies) followed by Goat Anti-Mouse IgG (H+L) Antibody, Polyclonal, FITC (#60138FI, STEMCELL Technologies), and Anti-Human CD45 Antibody, Clone HI30, APC (Catalog #60018AZ).

In vitro Expansion of CD34 positive cells. When there were too few of the above isolated CD34 positive cells from each sample (<200 cells) to make direct RNA purification for the subsequent mRNA expression measurement, the above prepared CD34 positive cells were further expanded by in vitro cell culture using StemSpan™ CD34+ Expansion Supplement (10X) in combination with StemSpan™ SFEM (#09650) medium. Assessment of CD34+ cells before and after culture was performed by flow cytometry using the following fluorochrome-conjugated antibody clones as mentioned above. The cells were harvested for mRNA purification or other potential applications after passage 2, such as for protein expression by western blotting.

Preparation of mRNA from MNCs or CD34 Positive Stem Cells. The average yield for MNCs isolation from peripheral blood was around 1×10⁶ MNCs/ml of blood, while umbilical cord blood had a yield of around 2×10⁶ MNCs/ml of blood. In MNCs, the peripheral blood had around 0.02% CD34 positive cells, while umbilical cord blood had around 0.5% CD34 positive cells. If 3 ml of the blood sample were used for isolation of CD34 positive cells, around 600 (3×1×10⁶×0.02%) of CD34 positive cells could be obtained from peripheral blood, and 30,000 (3×2×10⁶×0.5%) of CD34 positive cells could be obtained from umbilical cord blood. The isolated CD34 positive cells could be directly used for purification of total RNA using the RNeasy Plus Mini Kit or RNeasy Micro Kit (for isolation of RNA from <1000 cells, see attached instructions), and the final elute could be 30 μl using either RNase-free water or 10 mM Tris·Cl, pH 7.5. Purified RNA may be stored at −20° C. or −70° C. in water, and the concentration of RNA should be determined by measuring the absorbance at 260 nm (A260) in a spectrophotometer. To ensure significance, readings should be greater than 0.15. An absorbance of 1 unit at 260 nm corresponds to 40 μg of RNA per ml. This relation was valid only for measurements at a neutral pH. The ratio between the absorbance values at 260 and 280 nm gave an estimate of RNA purity. Pure RNA has an A260/A280 ratio of 1.9-2.1 in 10 mM TrisCl, pH 7.5.

Measurement of mRNA gene expression by RT reaction and real-time quantitative PCR. The above purified RNA was reverse transcribed using a Sensicript RT kit (Qiagen, see attached instruction). 1 μl of each cDNA was used to measure target genes. All of the primers were designed using Primer 3 Plus software with the Tm at 60° C., primer size of 21 bp, and product length in the range of 140-160 bp (see Table 1). The amplified products were confirmed with agarose gel. The real-time quantitative PCR was run on LightCycler® 480 Instrument II (Roche, Product No #: 05015243001, 384-well) with the Quantitect SYBR green PCR kit (Qiagen). The PCR was performed by denaturation at 95° C. for 15 min, followed by 40 cycles of denaturation at 94° C. for 15 s, annealing at 56° C. for 30 s, and extension at 72° C. for 30 s, respectively. β-actin or GAPDH was used as the housekeeping gene for transcript normalization, and the mean values were used to calculate relative transcript levels with the ΔΔCT method per instructions from Qiagen. In brief, the amplified transcripts were quantified by the comparative threshold cycle method using β-actin or GAPDH as a normalizer. Fold changes in gene mRNA expression were calculated as 2-ΔΔCT with CT=threshold cycle, ΔCT=CT(target gene)−CT(β-actin), and the ΔΔCT=ΔCT(experimental)−ΔCT (reference). The results are shown in Table 1 below.

Table 1. Sequences of Human Primers for the Real Time Quantitative PCR (qPCR)

TABLE 1
Sequences of human primers for the real time quantitative PCR (qPCR)

| | | Forward primer (5′→3′) | Reverse primer (5′→3′) | Change of |
|---|---|---|---|---|
| Gene | Accession # | (SEQ ID NO:_) | (SEQ ID NO :_ ) | Expression |
| β-actin | NM_001101 | GATGCAGAAGGAGATCACTGC (1) | ATACTCCTGCTTGCTGATCCA (2) | N/A |
| GAPDH | NM_002046 | CAAAAGGGTCATCATCTCTGC (3) | AGTTGTCATGGATGACCTTGG (4) | N/A |
| ERβ | NM_001040275 | ATGATGATGTCCCTGACCAAG (5) | ACATCAGCCCCATCATTAACA (6) | Down |
| ERα | NM_000125 | GATGATGGGCTTACTGACCAA (7) | AGACGAGACCAATCATCAGGA (8) | Down |
| GPER | NM_001505 | CTCCCTGCAAGCAGTCTTTC (9) | TCTGCTCAATGTACAGCCTCA (10) | Down |
| AR | NM_000044 | TCTTGTCGTCTTCGGAAATGT (11) | CACTGTCAGCTTCTGGGTTGT (12) | Down |
| OXT | NM_000915 | GCTGCCAGGAGGAGAACTAC (13) | CTGGGAGAAGGTGGCTTCC (14) | Down |
| OXTR | NM_000916 | CAACCCCTGGATCTACATGCT (15) | CAGGACAAAGGAGGACGAGTT (16) | Up |
| SOD2 | NM_000636 | GCCTACGTGAACAACCTGAAC (17) | TGAGGTTTGTCCAGAAAATGC (18) | Down |
| ERRα | NM_004451 | GAAGACAGCCCCAGTGAATG (19) | GACCACAATCTCTCGGTCAAA (20) | Down |
| PGR | NM_001202474 | AGCCCTAAGCCAGAGATTCAC (21) | CAGCAAAGAACTGGAGGTGTC (22) | Up |
| VDR | NM_000376 | ATCATTGCCATACTGCTGGAC (23) | GAGAAGCTGGGAGTGTGTCTG (24) | Down |
| RORA | NM_134261 | GGAGAAGTCAGCAAAGCAATG (25) | GACATTCGGCCAAATTTTACA (26) | Down |

### Example 2: Cutoff Values of Gene Expression Changes

This example expanded the testing of Example 1 to samples collected from more than 120 individuals. Further, for one of the genes, SOD2, which was confirmed as significantly downregulated in ASD patients, cut/off values were determined.

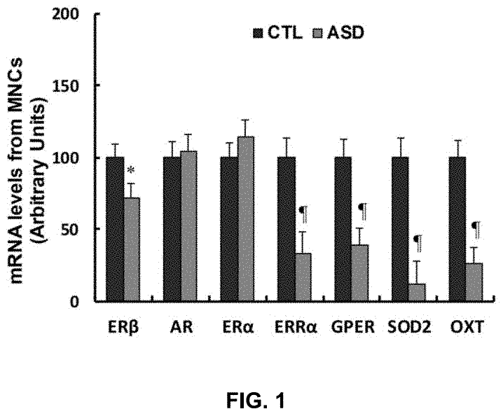

This experiment evaluated the mRNA expression of ERα, ERβ, SOD2, ERRα, OXT, OXTR, GPER, AR, PGR, RORA and VDR in MNCs isolated from peripheral blood of either control (CTL) or ASD patients. The most significant data are shown in FIG. 1. The results show that the ERβ mRNA from ASD group was decreased to 71.3% compared to the control (CTL) group, while the mRNA of ERRα, GPER, SOD2 and OXT from ASD group were decreased to 33.3%, 39.1%, 12.4% and 26.1%, respectively, compared to the CTL group. SOD2 mRNA levels had the most significant decrease in ASD group.

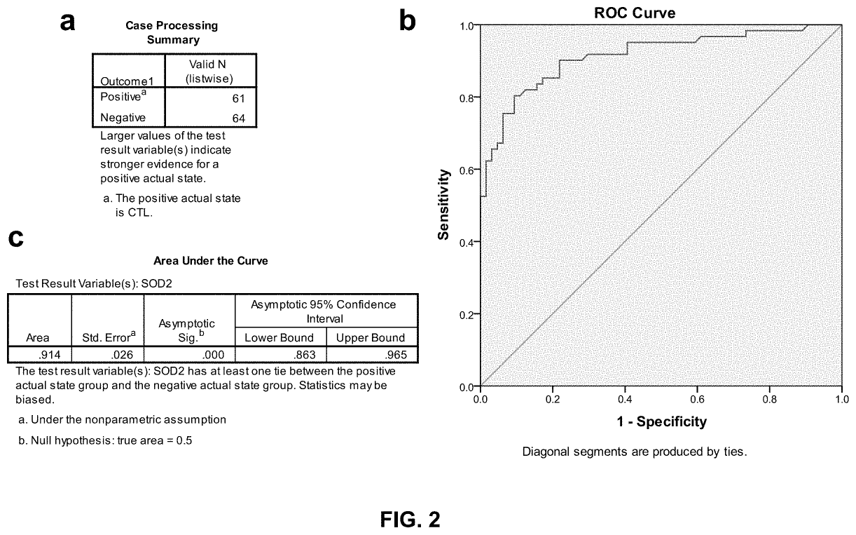

This example next established the ROC curve using the original SOD2 mRNA expression levels through SPSS 22 (see FIG. 2). The results show that there were 61 CTL cases and 64 ASD cases in total (see FIG. 2a), and the ROC curve was shown in FIG. 2b, and the Area under the curve was 0.914 (see FIG. 2c), which shows very good sensitivity and specificity for predicting in general.

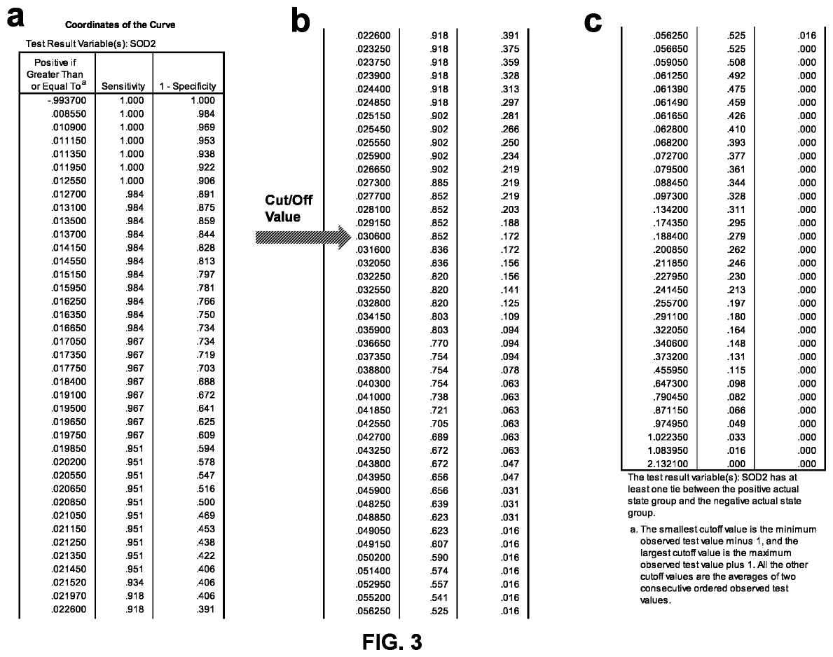

This example next established the Cut/Off Value for the diagnosis of ASD patients using the coordinates of the curve (see FIG. 3). As shown in FIG. 3, the Cut/Off Value was set as 0.0306 for the SOD2 mRNA level with 85% sensitivity and 83% specificity. <0.0306 in SOD2 mRNA was considered as the potential ASD patient.

The example demonstrates that the present technology is highly sensitive for the diagnosis of ASD patients based on the mRNA expression level of SOD2, ERRα, GPER, or OXT in MNCs of patient blood.

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1. A method for identifying and treating autism spectrum disorder (ASD) in a human patient in need thereof, comprising
measuring the mRNA expression levels of SOD2 (superoxide dismutase 2), GPER (G protein-coupled estrogen receptor 1), RORA (retinoic acid-related orphan receptor α), and PGR (progesterone receptor) in a mononuclear cell (MNC) or a CD34+ cell isolated from peripheral or umbilical cord blood of the human patient,
identifying the human patient as suffering from ASD when the mRNA expression levels of SOD2, GPER and RORA are decreased and the mRNA expression level of PGR is increased as compared to corresponding reference mRNA expression levels of SOD2, GPER, RORA and PGR from a reference human subject not suffering from an ASD, and
treating the human patient identified as suffering from ASD with a treatment selected from the group consisting of discrete trial training (DTT), pivotal response training (PRT), early intensive behavioral intervention (EMI), verbal behavior intervention (VBI), Developmental, Individual Differences, Relationship-Based Approach (DIR), Treatment and Education of Autistic and Related Communication-handicapped Children (TEACCH), The Picture Exchange Communication System (PECS), Sensory Integration Therapy, risperidone, a selective serotonin reuptake inhibitor (SSRI), an anti-anxiety medication, and a stimulant.

2. The method of claim 1, wherein the reference human subject is an actual reference human subject, or a virtual reference human subject created with pool data from one or more human subjects not suffering from an ASD.

3. The method of claim 1, further comprising isolating the MNC or CD34+ cell from the human patient.

4. The method of claim 1, wherein measurement is started within 8 hours following isolation of the cell from the human patient.

5. The method of claim 1, wherein the measurement is made from a cell population which includes at least 50% CD34+ cells.


SEQUENCE LISTING (26 sequences)

SEQ ID NO: 1 - DNA, 21 bp, synthetic construct
  misc_feature 1..21: note = Synthetic
  source 1..21: mol_type = other DNA
gatgcagaaggagatcactgc

SEQ ID NO: 2 - DNA, 21 bp, synthetic construct
  misc_feature 1..21: note = Synthetic
  source 1..21: mol_type = other DNA
atactcctgcttgctgatcca

SEQ ID NO: 3 - DNA, 21 bp, synthetic construct
  misc_feature 1..21: note = Synthetic
  source 1..21: mol_type = other DNA
caaaagggtcatcatctctgc

SEQ ID NO: 4 - DNA, 21 bp, synthetic construct
  misc_feature 1..21: note = Synthetic
  source 1..21: mol_type = other DNA
agttgtcatggatgaccttgg

SEQ ID NO: 5 - DNA, 21 bp, synthetic construct
  misc_feature 1..21: note = Synthetic
  source 1..21: mol_type = other DNA
atgatgatgtccctgaccaag

SEQ ID NO: 6 - DNA, 21 bp, synthetic construct
  misc_feature 1..21: note = Synthetic
  source 1..21: mol_type = other DNA
acatcagccccatcattaaca

SEQ ID NO: 7 - DNA, 21 bp, synthetic construct
  misc_feature 1..21: note = Synthetic
  source 1..21: mol_type = other DNA
gatgatgggcttactgaccaa

SEQ ID NO: 8 - DNA, 21 bp, synthetic construct
  misc_feature 1..21: note = Synthetic
  source 1..21: mol_type = other DNA
agacgagaccaatcatcagga

SEQ ID NO: 9 - DNA, 20 bp, synthetic construct
  misc_feature 1..20: note = Synthetic
  source 1..20: mol_type = other DNA
ctccctgcaagcagtctttc

SEQ ID NO: 10 - DNA, 21 bp, synthetic construct
  misc_feature 1..21: note = Synthetic
  source 1..21: mol_type = other DNA
tctgctcaatgtacagcctca

SEQ ID NO: 11 - DNA, 21 bp, synthetic construct
  misc_feature 1..21: note = Synthetic
  source 1..21: mol_type = other DNA
tcttgtcgtcttcggaaatgt

SEQ ID NO: 12 - DNA, 21 bp, synthetic construct
  misc_feature 1..21: note = Synthetic
  source 1..21: mol_type = other DNA
cactgtcagcttctgggttgt

SEQ ID NO: 13 - DNA, 20 bp, synthetic construct
  misc_feature 1..20: note = Synthetic
  source 1..20: mol_type = other DNA
gctgccaggaggagaactac

SEQ ID NO: 14 - DNA, 19 bp, synthetic construct
  misc_feature 1..19: note = Synthetic
  source 1..19: mol_type = other DNA
ctgggagaaggtggcttcc

SEQ ID NO: 15 - DNA, 21 bp, synthetic construct
  misc_feature 1..21: note = Synthetic
  source 1..21: mol_type = other DNA
caacccctggatctacatgct

SEQ ID NO: 16 - DNA, 21 bp, synthetic construct
  misc_feature 1..21: note = Synthetic
  source 1..21: mol_type = other DNA
caggacaaaggaggacgagtt

SEQ ID NO: 17 - DNA, 21 bp, synthetic construct
  misc_feature 1..21: note = Synthetic
  source 1..21: mol_type = other DNA
gcctacgtgaacaacctgaac

SEQ ID NO: 18 - DNA, 21 bp, synthetic construct
  misc_feature 1..21: note = Synthetic
  source 1..21: mol_type = other DNA
tgaggtttgtccagaaaatgc

SEQ ID NO: 19 - DNA, 20 bp, synthetic construct
  misc_feature 1..20: note = Synthetic
  source 1..20: mol_type = other DNA
gaagacagccccagtgaatg

SEQ ID NO: 20 - DNA, 21 bp, synthetic construct
  misc_feature 1..21: note = Synthetic
  source 1..21: mol_type = other DNA
gaccacaatctctcggtcaaa

SEQ ID NO: 21 - DNA, 21 bp, synthetic construct
  misc_feature 1..21: note = Synthetic
  source 1..21: mol_type = other DNA
agccctaagccagagattcac

SEQ ID NO: 22 - DNA, 21 bp, synthetic construct
  misc_feature 1..21: note = Synthetic
  source 1..21: mol_type = other DNA
cagcaaagaactggaggtgtc

SEQ ID NO: 23 - DNA, 21 bp, synthetic construct
  misc_feature 1..21: note = Synthetic
  source 1..21: mol_type = other DNA
atcattgccatactgctggac

SEQ ID NO: 24 - DNA, 21 bp, synthetic construct
  misc_feature 1..21: note = Synthetic
  source 1..21: mol_type = other DNA
gagaagctgggagtgtgtctg

SEQ ID NO: 25 - DNA, 21 bp, synthetic construct
  misc_feature 1..21: note = Synthetic
  source 1..21: mol_type = other DNA
ggagaagtcagcaaagcaatg

SEQ ID NO: 26 - DNA, 21 bp, synthetic construct
  misc_feature 1..21: note = Synthetic
  source 1..21: mol_type = other DNA
gacattcggccaaattttaca